(19) United States
(12) Patent Application Publication
(10) Pub. No.: US 2005/0272102 A1
(43) Pub. Date: Dec. 8, 2005

(54) Method for diagnosis of prostate cancer

(75) Inventors: John C. Mauck, Rochester, NY (US); Paul N. Schnipelsky, Pulteney, NY (US); James F. Devanney, Winsted, CT (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Egenix, Inc., Millbrook, NY (US)

(21) Appl. No.: 11/127,042

(22) Filed: May 11, 2005

Related U.S. Application Data
(60) Provisional application No. 60/570,416, filed on May 12, 2004.

(51) Int. Cl. G01N 33/574

(52) U.S. Cl. 435/7.23

(57) ABSTRACT

Methods and kits useful for diagnosis of prostate cancer are disclosed, based on levels of Human Carcino Antigen in semen or other biological samples.

### RELATED APPLICATION(S)

This application claims the benefit of U.S. Provisional Application No. 60/570,416 filed on May 12, 2004. The entire teachings of the above application is incorporated herein by reference.

### BACKGROUND OF THE INVENTION

Prostate cancer typically afflicts aging males, but it can afflict males of all ages. Worldwide, prostate cancer is the fourth most prevalent cancer in men. In North America and Northern Europe, it is by far the most common male cancer and is the second leading cause of cancer death. Early diagnosis of prostate cancer in patients reduces the likelihood of death.

Most prostate cancers initially occur in the peripheral zone of the prostate gland, away from the urethra. Tumors within this zone may not produce any symptoms and, as a result, most men with early-stage prostate cancer will not present clinical symptoms of the disease until significant progression has occurred. Tumor progression into the transition zone of the prostate may lead to urethral obstruction, thus producing the first symptoms of the disease. However, these clinical symptoms are indistinguishable from the common non-malignant condition of benign prostatic hyperplasia (BPH), the prevalence of which in a population of suspect patients is many times greater than that of prostate cancer. Early detection and diagnosis of prostate cancer currently relies on digital rectal examinations (DRE), prostate specific antigen (PSA) measurements, transrectal ultrasonography (TRUS), and transrectal needle biopsy (TRNB). At present, serum PSA measurement in combination with DRE represent the leading tool used to detect and diagnose prostate cancer.

Conventionally, prostate cancer is diagnosed using PSA as a marker. In general, PSA levels above 4 ng/ml are suggestive of prostate cancer while levels above 10 ng/ml are highly suggestive of prostate cancer. However, if the cancer is in its early stages, some prostate cancer patients exhibit normal PSA levels at the time of diagnosis. Since conventional PSA tests detect abnormal levels of PSA, conventional PSA tests may not be able to detect the presence of prostate cancer if it is in its early stages. This results in a false negative diagnosis. The inability of conventional PSA tests to diagnose the presence of prostate cancer in some instances (e.g., in the early stages of the disease) can be detrimental to the patient. Moreover, PSA is not a disease-specific marker, as elevated levels of PSA are detectable in a large percentage of patients with BPH, as well as in other nonmalignant disorders and in some normal men, a factor which significantly limits the diagnostic specificity of this marker. Further confusing the situation is the fact that serum PSA elevations may be observed without any indication of disease from DRE, and visa-versa.

Thus, although the serum PSA assay has been a very useful tool, its specificity and general utility is widely regarded as lacking, in that it provides significant numbers of false positive and false negative results. Better diagnosis will result from the discovery of disease markers that can be used alone or in combination to increase the specificity and selectivity of diagnostic tests for prostate cancer.

### SUMMARY OF THE INVENTION

The present invention relates to the discovery that measurement of Human Carcino Antigen (HCA) in human semen and other human biological samples containing prostatic seminal fluid, particularly ejaculate, provides a sensitive and accurate diagnostic test for prostate cancer. Measurement of HCA in semen has also been discovered to be superior to measuring HCA in other body fluids (e.g., serum, plasma) for diagnosis or detection of prostate cancer.

Accordingly, the present invention provides methods for diagnosis of prostate cancer in a human subject comprising determining the level of HCA in a semen sample from the subject; and comparing the level determined to the level of HCA in a control semen sample. As used herein, a control semen sample is obtained from a normal subject, age-matched and demographically matched to the subject undergoing the diagnostic analysis. As used herein a “normal subject” does not have prostate cancer. In a particular embodiment, the level of HCA in a semen sample is determined by a competitive immunoassay procedure. In another embodiment, the level of HCA in a semen sample is determined by a sandwich assay procedure. An elevated level of HCA in the semen sample relative to the control is indicative of prostate cancer.

In a particular embodiment, the invention provides methods for diagnosis of prostate cancer in a human subject comprising (a) contacting a semen sample from the subject with an antibody or antigen-binding fragment thereof which is specific for HCA under conditions sufficient for binding between HCA and the antibody or antigen-binding fragment (formation of an immunocomplex between HCA and the antibody); (b) assaying for binding of HCA to the antibody or antigen-binding fragment (formation of immunocomplex); (c) determining the level of HCA bound to said antibody or antigen-binding fragment; and (d) comparing the level of bound HCA determined in step (c) to the level of HCA bound to the antibody in a control semen sample. In a particular embodiment, binding of HCA to antibody or antigen-binding fragment (formation of immunocomplex) is determined by a competitive immunoassay procedure. In another embodiment, binding of HCA to antibody or antigen-binding fragment (formation of immunocomplex) is determined by a sandwich assay procedure. Binding of HCA to antibody or antigen-binding fragment (formation of immunocomplex) reflects the presence of HCA in the sample. The presence of an elevated level of HCA bound to antibody relative to the control is indicative of prostate cancer.

The invention also provides kits for diagnosis of prostate cancer. In one embodiment, the kit comprises an antibody or antigen-binding fragment thereof which binds to HCA and suitable ancillary reagents. In a particular embodiment, the kit comprises: (a) an immobilized antigen that is comprised of either HCA, epiglycanin, an idiotypic antibody to the detecting antibody (AE3) or a surrogate antigen that has a similar affinity as HCA to AE3; (b) a suitable immobilized phase (e.g., micro titer plates, insoluble polymeric beads or particles) that can be washed and separated from a reaction mixture and are suitable for the immobilization of the antigen of (a); (c) a specific antibody AE3 with high affinity to HCA that can be detected using a detection method (e.g., radiation, colorimeteric, enzymatic, chemiluminecence, etc.), either directly or indirectly; (d) a series of calibration material (calibrators) comprised of materials that emulate HCA in patient samples that can be used to establish an appropriate response curve to map detection signal into concentration of HCA; and (e) any required blocking agents and buffers that inhibit nonspecific binding or any other signal generating reactions that are unrelated to HCA concentration. The calibrators of step (d) are stable over the useful lifetime of the kit.

### BRIEF DESCRIPTION OF THE DRAWINGS

FIG. 1A is a table of data from a 35 patient study. Semen samples from 35 semen donors were analyzed for HCA and PSA.

FIG. 1B is a graphic correlation of HCA and PSA values for 35 semen donors (35 patient study). The results show that there is no correlation between HCA and PSA.

FIG. 2A shows the patient population statistical values for the HCA and PSA determinations from 35 semen samples (35 patient study).

FIG. 2B shows the receiver operator characteristic (ROC) curves for HCA and PSA values for 35 semen donors (35 patient study).

FIGS. 2C and 2D show the clinical performance of HCA and PSA for 35 semen donors (35 patient study).

FIG. 3A shows the patient population statistical values for the HCA determinations from 84 semen samples (84 patient study).

FIG. 3B shows the receiver operator characteristic (ROC) curve for HCA values for 84 semen donors (84 patient study).

FIGS. 3C and 3D show the clinical performance of HCA for 84 semen donors (84 patient study).

FIG. 4A shows the patient population statistical values for the HCA determinations from 433 serum samples.

FIG. 4B shows the receiver operator characteristic (ROC) curve for HCA values for 433 serum samples.

FIG. 4C to 4H show the clinical performance of HCA for 433 serum samples.

FIG. 5A shows the patient population statistical values for the PSA and HCA determinations from 358 serum samples.

FIG. 5B shows the receiver operator characteristic (ROC) curve for PSA and HCA values for 358 serum samples.

FIGS. 5C to 5J show the clinical performance of PSA and HCA for 358 serum samples.

FIG. 6 is an example of a semen calibration curve that can be used to predict HCA concentration in ejaculate. Calibrators HCA concentration were determined by COD competitive assay, except that the concentrations of the constituents were at levels appropriate for semen. Ejaculate was diluted to fit on the curve. The curve is optical density of signal versus the log of the concentration of the calibrator.

FIG. 7 is a bar graph showing the results of an ELISA in which four monoclonal antibodies to HCA with low cross-reactivity with MUC6 were compared with a monoclonal antibody to HCA known to strongly cross-react with MUC6.

### DETAILED DESCRIPTION OF THE INVENTION

HCA is a complex glycoprotein molecule distinctively expressed on human carcinomas. The molecular weight of HCA is in excess of 750 kDa, with over 75% of the molecule comprised of carbohydrate moieties characteristic of mucin-type glycoproteins. The carbohydrate moiety consists of a relatively high proportion of sialic acid, galactose, and N-acetylgalactosamine residues (e.g., at least 50% of the carbohydrate residues are sialic acid, galactose, or N-acetylgalactosamine residues). The isoelectric point of HCA is below pH 3.0 and is generally insoluble in aqueous fluids (e.g., a phosphoric acid or a HCl solution). HCA has been described in the art (see, e.g., U.S. Pat. No. 5,808,005, issued to John F. Codington et al.; U.S. Pat. No. 5,693,763, issued to John F. Codington et al.; U.S. Pat. No. 5,545,532, issued to John F. Codington et al., the teachings of each of which are incorporated herein by reference).

The present invention relates to the discovery that measurement of HCA in human semen and other human biological samples containing prostatic seminal fluid, particularly ejaculate, provides a sensitive and accurate diagnostic test for prostate cancer. Measurement of HCA in semen has also been discovered to be superior to measuring HCA in other body fluids (e.g., serum, plasma) for diagnosis or detection of prostate cancer.

For example, HCA levels in blood serum are approximately 1/100 of the corresponding measurement in semen and other human biological samples containing prostatic seminal fluid. This increase in HCA level, undiluted by other organ or tissue contributions or secretions, allows semen and seminal fluid to be analyzed faster and with less interference from irrelevant endogenous components of the sample. As used herein, “irrelevant endogenous components” that can interfere with the analyses described herein include species from other organs and tissues that are cross-reactive with the detection antibody (anti-HCA antibody). Such “irrelevant endogenous components” are present in blood, serum, plasma and lymphatic fluid. Natural degradation products of HCA, which are present in blood, serum, plasma and urine, can also inhibit the activity of HCA present in these fluids and thus, interfere with a diagnosis or detection of prostate cancer. In the present invention, other organs and functions do not directly add interferences to the semen or seminal fluid sample since the sample comes primarily from the organ to be diagnosed. Another advantage of measuring HCA in semen is that immunoassays can be performed that have a longer dynamic range requiring fewer samples to have to be serially diluted to bring them within the linear analytical range of the assay in contrast to HCA in serum.

Accordingly, the present invention provides methods for diagnosis of prostate cancer in a human subject comprising determining the level of HCA in a semen sample from the subject; and comparing the level determined to the level of HCA in a control semen sample. A control semen sample is obtained from a normal subject, age-matched and demographically matched to the subject undergoing the diagnostic procedure for prostate cancer described herein. As used herein, a “normal subject” does not have prostate cancer. An elevated level of HCA in the semen sample relative to the control is indicative of prostate cancer.

In a particular embodiment, the invention provides methods for diagnosis of prostate cancer in a human subject comprising (a) contacting a semen sample from the subject with an antibody or antigen-binding fragment thereof which is specific for HCA under conditions sufficient for binding between HCA and the antibody or antigen-binding fragment (formation of an immunocomplex between HCA and the antibody); (b) assaying for binding of HCA to the antibody or antigen-binding fragment (formation of immunocomplex); (c) determining the level of HCA bound to said antibody or antigen-binding fragment; and (d) comparing the level of bound HCA determined in step (c) to the level of HCA bound to the antibody in a control semen sample. Binding of HCA to antibody or antigen-binding fragment (formation of immunocomplex) reflects the presence of HCA in the sample. The presence of an elevated level of HCA bound to antibody relative to the control is indicative of prostate cancer.

Immunoassays are any assays that can detect the binding (or absence of binding) of an antigen to an antibody or antigen-binding fragment and quantitate the presence of the antigen in the sample. Examples of suitable immunoassays include sandwich assays, radioimmunoassays and, preferably, competitive inhibition assays. The use of the term “antigen” or “inhibitor” in the context of a reagent in the assay is intended to include HCA, as well as functional variants and portions of HCA. An inhibitor, as used herein, refers to an antigen that is immunologically cross-reactive with HCA.

Functional variants of HCA include functional fragments, functional mutant proteins and/or functional fusion proteins which can be produced using suitable methods (e.g., mutagenesis (e.g., chemical mutagenesis, radiation mutagenesis), recombinant DNA techniques). A functional variant of HCA is a protein or polypeptide which has at least one function characteristic of HCA, as described herein, such as a binding activity.

Generally, fragments or portions of HCA include those having a deletion (i.e., one or more deletions) of an amino acid (i.e., one or more amino acids) relative to the native (wildtype) HCA, respectively (such as N-terminal, C-terminal or internal deletions). Fragments or portions in which only contiguous amino acids have been deleted or in which non-contiguous amino acids have been deleted relative to native (wildtype) HCA are also envisioned.

Mutant HCA include natural or artificial variants of a HCA differing by the addition, deletion and/or substitution of one or more contiguous on non-contiguous amino acid residues. Such mutations can occur at one or more sites on a protein, for example a conserved region or nonconserved region.

Fusion proteins encompass polypeptides comprising a HCA or variants thereof, as a first moiety, linked via a covalent bond (e.g., peptide bond) to a second moiety not occurring in the HCA as found in nature. Thus, the second moiety can be linked to a first moiety at a suitable position, for example, the N-terminus, the C-terminus or internally.

In a radioimmunoassay (RIA), the amount of antigen present in a sample is measured indirectly employing a limited amount of antibody (or antigen-binding fragment) to compete for labeled antigen. In an IRMA (immunoradiometric assay), antigen is assayed directly by reacting the antigen with excess labeled antibody (or antigen-binding fragment).

In one class of IRMA assays, the unknown antigen is insolubilized and reacted with labeled antibody (or antigen-binding fragment). When the antigen is insolubilized by reaction with solid-phase antibody (or antigen-binding fragment), the assay is termed a “two-site IRMA”, “junction test”, or “sandwich assay”. Sandwich assays are further classified according to their methodology as forward, reverse or simultaneous sandwich assays.

In a forward sandwich immunoassay, a sample containing the antigen can be first incubated with a solid-phase immunoadsorbent containing immobilized antibody (or antigen-binding fragment). Incubation is continued for a sufficient period of time to allow antigen in the sample to bind to immobilized antibody (or antigen-binding fragment) on the solid-phase immunoadsorbent. The solid-phase immunoadsorbent can then be separated from the incubation mixture and washed to remove excess antigen and other substances which also may be present in the sample. The solid-phase immunoadsorbent containing antigen (if any) bound to immobilized antibody (or antigen-binding fragment) can be subsequently incubated with labeled antibody (or antigen-binding fragment) capable of binding to the antigen. After the second incubation, another wash is performed to remove unbound labeled antibody (or antigen-binding fragment) from the solid-phase immunoadsorbent thereby removing non-specifically bound labeled antibody (or antigen-binding fragment). Labeled antibody (or antigen-binding fragment) bound to the solid-phase immunoadsorbent is then detected and the amount of labeled antibody (or antigen-binding fragment) detected can serve as a direct measure of the amount of antigen present in the sample. Such forward sandwich assays are described in the patent literature, and in particular, in U.S. Pat. Nos. 3,867,517 and 4,012,294, issued to Chung-Mei Ling, which are incorporated herein by reference.

In a reverse sandwich assay, a sample can be incubated with labeled antibody (or antigen-binding fragment) after which the solid-phase immunoadsorbent containing immobilized antibody (or antigen-binding fragment) is added and incubated. A washing step can be performed after the second incubation period. A reverse sandwich assay has been described in the patent literature in U.S. Pat. No. 4,098,876, issued to Roger N. Piasio et al.

In a simultaneous sandwich assay, a sample can be incubated simultaneously in one step with both an immunoadsorbent containing immobilized antibody (or antigen-binding fragment) for the antigen and labeled antibody (or antigen-binding fragment) for the antigen. Thereafter, labeled antibody (or antigen-binding fragment) bound to the immunoadsorbent can be detected as an indication of the amount of antigen present in the sample. A simultaneous sandwich assay has been described in the patent literature in U.S. Pat. No. 4,837,167, issued to Hubert J. P. Schoemaker et al.

Many solid-phase immunoadsorbents can be employed. Well-known immunoadsorbents include beads formed from glass polystyrene, polypropylene, dextran, and other materials. Preferably, the solid support is a plate, stick, tube or well formed from or coated with such materials; etc. The antibody (or antigen-binding fragment) can be either covalently or physically bound to the solid-phase immunoadsorbent by techniques such as covalent bonding via an amide or ester linkage or adsorption. Many other suitable solid-phase immunoadsorbents and methods for immobilizing antibodies (or antigen-binding fragments) thereon are known in the art.

A competitive inhibition immunoassay can be employed to determine the presence of an antigen in a sample by measuring the inhibition of formation of a competitive inhibitor-antibody (or competitive inhibitor-antigen-binding fragment) complex, one of which is typically bound and the other of which is typically labeled, by free antigen in the sample. In addition, a typical quantitative immunoassay kit can include a standardized sample of pure inhibitor, such as an antigen, so that a reference solution can be run together with the sample to minimize sampling errors and to assure precision.

Competitive immunoassays (e.g., radioimmunoassay (RIA), enzyme-linked immunoadsorbant assay (ELISA)) are used to detect and quantitate the presence of antigen in a sample by determining the extent of inhibition by the antigen of a competitive inhibitor/antibody (or competitive inhibitor/antigen-binding fragment) reaction. Typically, either the inhibitor or the antibody (or antigen-binding fragment) is bound to a solid support (as described above), while the other component of the pair is labeled in some fashion to render it detectable. Methods that are used to detect and quantitate the presence of antigen in a sample are also referred to as serologic diagnostic methods.

Labels are well known in the art and include, e.g., radionuclides (e.g., Iodine-125, Iodine-131, Indium-111, Bismuth-210), enzymes which produce an absorptive or fluorescent detector group when reacted with a specific substrate (e.g., horseradish peroxidase, N-methylumbelliferone-o-D-galactosidase), dyes (chromophores), fluorescent compounds (e.g., fluorescein, rhodamine, phycoerythrin, cyamine dyes, other compound emitting fluorescence energy), electron dense compounds (e.g., gold and ferric chloride compounds). Biotin/avidin labeling systems can also be used. Coupled assays can also be used for detecting labels.

The label may be directly linked to the component (the inhibitor or antibody) or may be bound to it indirectly, e.g., by attaching the label to another molecule capable of recognizing a component of the antigen/antibody pair. For example, an antibody (or antigen-binding fragment) can be indirectly labeled by attaching an enzyme, fluorescent marker or radionuclide to an isotype-specific antibody which recognizes the non-variable region of the antigen-specific antibody (or antigen-binding fragment). In another embodiment, the label can be attached to an antibody (or antigen-binding fragment) which recognizes an available epitope of the antigen after it has been bound to the specific antibody (or antigen-binding fragment). Many other variants of this broad concept are possible and known in the art.

In one preferred embodiment, the label is a dye (such as, nitrophenyl) attached to the unbound component or reagent (unbound inhibitor or antibody) via a phosphate linker. After incubation of the labeled component with the immobilized binding partner, the presence of binding can then be determined by subjecting the solid support to a phosphatase enzyme, causing hydrolysis of the dye. The presence (and amount) of the dye can then be measured by absorbance, indicating the amount of binding of the two components.

In each assay, the sample, antibody (or antigen-binding fragment) and, optionally, the inhibitor is incubated under conditions and for a period of time sufficient to allow antigen to bind to the antibody (or antigen-binding fragment), i.e., under conditions suitable for the formation of a complex between the antigen and antibody (or antigen-binding fragment). In general, it is usually desirable to provide incubation conditions sufficient to bind as much antigen or inhibitor as possible because this maximizes the binding of labeled antibody or antigen-binding fragment) to the antigen thereby increasing the signal. Suitable temperatures are generally below the temperature at which denaturation can occur.

The presence of an increased (elevated) level of HCA reactivity in a semen sample obtained from a subject can be indicative of malignancy associated with prostate cancer. Measurement of HCA in a semen sample can provide early diagnosis of prostate cancer and the opportunity for early treatment.

The level of HCA measured in a semen sample provides a means for monitoring the course of the cancer therapy, including surgery, chemotherapy, radiation therapy. The presence of HCA is directly related to the presence of cancer. If patients are undergoing successful treatment and the cancer is disappearing, the level of HCA is reduced. That is, the course of cancer therapy can be monitored by assessing HCA immunoreactivity in a semen sample from a subject. By correlating the level of HCA with the severity of disease, the level of HCA can be used to indicate successful removal of the primary tumor and/or metastases, and the effectiveness of other therapies over time. A decrease in the level over time indicates a reduced tumor burden in the patient. In contrast, no change or an increase indicates ineffectiveness of therapy or the continued growth of the tumor.

Suitable antibodies, and antigen-binding fragments thereof, for use in determining the presence of HCA bind to the antigen HCA. Such antibodies include antibodies to HCA, as well as antibodies to epiglycanin that cross-react and bind HCA.

Antibodies to HCA and methods for their production have been described in the art (see, e.g., U.S. Pat. No. 5,808,005; U.S. Pat. No. 5,693,763; U.S. Pat. No. 5,545,532, the teachings of which are incorporated herein by reference).

Antibodies to epiglycanin and methods for their production have also been described in the art. For example, monoclonal antibodies to epiglycanin and methods for their production are described, for example, in U.S. Pat. No. 4,837,171, issued to John F. Codington; U.S. Pat. No. 5,545,532, issued to John F. Codington et al.; and Haavik et al., Glycobiology, 2:217-224 (1992), the teachings of all of which are entirely incorporated herein by reference. Hybridomas producing anti-murine epiglycanin antibodies, AE-1, AE-3 and AE-4, have been deposited with the American Type Culture Collection (ATCC), P.O. Box 1549, Manassas, Va. 20108 USA. For example, the hybridoma HAE-1 (producing monoclonal antibody AE-1) was deposited at the ATCC under accession no. HB-9466. The hybridoma HAE-3 (producing monoclonal antibody AE-3) was deposited at the ATCC under accession no. HB-9467. The hybridoma HAE-4 (producing monoclonal antibody AE-4) was deposited at the ATCC under accession no. HB-9468. Monoclonal antibody AE-3 cross-reacts and binds with HCA (see, e.g., U.S. Pat. No. 5,808,005; U.S. Pat. No. 5,693,763; U.S. Pat. No. 5,545,532, all issued to John F. Codington et al.). Similar antibodies can be prepared by known methods. Epiglycanin can be obtained, for example, as described in U.S. Pat. No. 4,837,171, issued to John F. Codington, the teaching of which is entirely incorporated herein by reference. Where an antibody is produced employing epiglycanin, or an immunogenic fragment thereof, as the immunogen, the resulting antibodies are screened for their ability to cross-react and bind HCA.

Antibodies can be polyclonal or monoclonal, and the term “antibody” is intended to encompass both polyclonal and monoclonal antibodies. The terms polyclonal and monoclonal refer to the degree of homogeneity of an antibody preparation, and are not intended to be limited to particular methods of production. The term “antibody”, as used herein, also encompasses functional fragments of antibodies, including fragments of human, chimeric, humanized, primatized, veneered or single chain antibodies. Functional fragments include antigen-binding fragments specific for HCA. Antigen-binding fragments specific for HCA include, but are not limited to, Fab, Fab′, F(ab′)₂ and Fv fragments. Such fragments can be produced by enzymatic cleavage or recombinant techniques. For example, papain or pepsin cleavage can generate Fab or F(ab′)₂ fragments, respectively. Other proteases with the requisite substrate specificity can also be used to generate Fab or F(ab′)₂ fragments. Antibodies can also be produced in a variety of truncated forms using antibody genes in which one or more stop codons has been introduced upstream of the natural stop site. For example, a chimeric gene encoding a F(ab′)₂ heavy chain portion can be designed to include DNA sequences encoding the CH₁ domain and hinge region of the heavy chain.

Single chain antibodies, and chimeric, humanized or primatized (CDR-grafted), or veneered antibodies, as well as chimeric, CDR-grafted or veneered single chain antibodies, comprising portions derived from different species, and the like are also encompassed by the present invention and the term “antibody”. The various portions of these antibodies can be joined together chemically by conventional techniques, or can be prepared as a contiguous protein using genetic engineering techniques. For example, nucleic acids encoding a chimeric or humanized chain can be expressed to produce a contiguous protein. See, e.g., Cabilly et al., U.S. Pat. No. 4,816,567; Cabilly et al., European Patent No. 0 125 023 B1; Boss et al., U.S. Pat. No. 4,816,397; Boss et al., European Patent No. 0 120 694 B1; Neuberger et al., International Publication No. WO86/01533; Neuberger et al., European Patent No. 0 194 276 B1; issued to Winter et al., U.S. Pat. No. 5,225,539; issued to Winter et al., European Patent No. 0 239 400 B1; Queen et al., European Patent No. 0 451 216 B1; and Padlan et al., EP 0 519 596 A1. See also, Newman et al., BioTechnology, 10: 1455-1460 (1992), regarding primatized antibody, and Ladner et al., U.S. Pat. No. 4,946,778 and Bird et al., Science, 242:423-426 (1988)) regarding single chain antibodies.

An “antigen” is a molecule or a portion of a molecule capable of being bound by an antibody which is additionally capable of inducing an animal to produce antibody capable of binding to an epitope of that antigen. An antigen can have one or more than one epitope.

The term “epitope” is meant to refer to that portion of the antigen capable of being recognized by and bound by an antibody at one or more of the antibody's antigen binding region. Epitopes usually consist of chemically active surface groupings of molecules such as amino acids or sugar side chains and have specific three dimensional structural characteristics as well as specific charge characteristics.

To block MUC6 from binding with an anti-HCA antibody in the sample immunoassay, an agonist or antagonist that blocks or interferes with MUC6 binding to anti-HCA antibodies, but does not block or interfere with HCA binding to anti-HCA antibodies, can be added to the sample before or at the same time the assay is performed. In a particular embodiment, MUC6 binding can be blocked by adding an anti-MUC6 antibody to the sample before or at the same time the sample immunoassay is performed. An example of an anti-MUC6 antibody is NCL MUC6 from Vector Laboratories, Inc. (Burlingame, Calif.). Other MUC6 antagonists and agonists, including other anti-MUC6 antibodies, are known and described in the art. In another embodiment, anti-HCA antibodies that are not cross-reactive with MUC6 can be used in immunoassays for detecting HCA. Such antibodies can be generated using HCA isolated either from prostate cancer tissues or from prostate tumor cell lines.

Kits for use in detecting the presence of HCA in a semen sample can also be prepared. Such kits can include an antibody or antigen-binding fragment which binds HCA, as well as one or more ancillary reagents suitable for detecting the presence of a complex between the antibody or fragment and HCA. The antibody or antigen binding fragment compositions can be provided in lyophilized form, either alone or in combination with additional antibodies specific for other epitopes. The antibodies or antigen-binding fragments, which can be labeled or unlabeled, can be included in the kits with adjunct ingredients (e.g., buffers, such as Tris, phosphate and carbonate, stabilizers, excipients, biocides and/or inert proteins, e.g., bovine serum albumin). For example, the antibodies or antigen-binding fragments can be provided as a lyophilized mixture with adjunct ingredients, or adjunct ingredients can be separately provided for combination by the user. Where a second antibody or antigen-binding fragment which binds HCA is employed, such antibody or fragment can be provided in the kit, for instance in a separate vial or container. The second antibody or fragment, if present, is typically labeled, and can be formulated in an analogous manner with the antibody or fragment formulations described above. The components (e.g., antibody, ancillary reagent) of the kit can be packaged separately or together within suitable containment means (e.g., bottle, box, envelope, tube). When the kit comprises a plurality of individually packaged components, the individual packages can be contained within a single larger containment means (e.g., bottle, box, envelope, tube). In a particular embodiment, the kit comprises: (a) an immobilized antigen that is comprised of either HCA, epiglycanin, an idiotypic antibody to the detecting antibody (AE3) or a surrogate antigen that has a similar affinity as HCA to AE3; (b) a suitable immobilized phase (e.g., micro titer plates, insoluble polymeric beads or particles) that can be washed and separated from a reaction mixture and are suitable for the immobilization of the antigen of (a); (c) a specific antibody AE3 with high affinity to HCA that can be detected using a detection method (e.g., radiation, colorimeteric, enzymatic, chemiluminecence, etc.), either directly or indirectly; (d) a series of calibration material (calibrators) comprised of materials that emulate HCA in patient samples that can be used to establish an appropriate response curve to map detection signal into concentration of HCA; and (e) any required blocking agents and buffers that inhibit nonspecific binding or any other signal generating reactions that are unrelated to HCA concentration. The calibrators of step (d) are stable over the useful lifetime of the kit.

The present invention will now be illustrated by the following examples, which are not to be considered limiting in any way.

### EXAMPLES

### Example 1

### Human Carcinoma Antigen Measured In Ejaculate To Distinguish Between Prostatic Carcinoma and Benign Prostatic Hyperplasia

Introduction

Human Carcinoma Antigen (HCA) is a cell surface mucin protein recognized by antibodies raised against epiglycanin from mouse mammary carcinoma cells. HCA level is increased in sera and tissue from patients with Prostatic Carcinoma (PC). The objective of this study was to determine if levels of HCA in ejaculate can be used to distinguish between Benign Prostatic Hyperplasia (BPH) and PC. Serum and tissue HCA have been previously reported (J. Urology, 161(4 Suppl.: 209 (1999); Li, R. et. al., Modern Pathology, 16(1):159A (2003)). Ejaculate has not been previously studied.

Methods

Ejaculates were collected from patients who were to undergo prostate biopsy, patients who were normal and age matched, and patients who were undergoing routine fertility examination in a fertility/andrology laboratory. Samples were frozen until they were examined by a competitive immunoassay for HCA in triplicate. The method was similar to the competitive serum assay reported by Codington, J. C. et. al. (J. Natl. Cancer Inst., 73(5):1029-1037 (1984)) with the exception of the sample dilution levels. PSA levels were measured on corresponding serum samples. Data were analyzed with Receiver Operator Curve (ROC) methods.

Results

Patient samples were categorized (9 cancer and 75 non-cancer) and ROC analysis provided the following results: A.U.C. 0.929, cutoff 190, Sensitivity 100%, Specificity 83%. Age and HCA level and PSA versus HCA level were uncorrelated (R2=6E-06, R2=0.0024). Levels of HCA were substantially higher in ejaculate than serum (50 to 10×).

Conclusions

HCA from ejaculate is a useful and practical marker to aid in the diagnosis of prostatic carcinoma. HCA from ejaculate is superior to serum PSA as a prostatic marker since it is not correlated with age. The results in this study demonstrate that measurement of HCA in ejaculate provides excellent sensitivity and specificity for diagnosis of prostatic carcinoma.

### Example 2

### Patient Studies: Analysis of Semen Samples

Two studies on semen samples for HCA and PSA, designated “35 Patient Study” and “84 Patient Studies”, were conducted.

The 35 Patient Study samples were from a urology clinic. All except four samples were from biopsied (non-cancer) patient samples.

Some of the patient samples for the 84 Patient Study were from the University of Rochester Andrology Clinic; these samples were anonymous samples from patients undergoing normal fertility studies or preserving their sperm prior to treatment for cancer. Other non-cancer patient samples were from a urology clinic or normal volunteers. The other cancer samples were from the urology clinic. Some of the non-cancers were clinically cancer free but not biopsied.

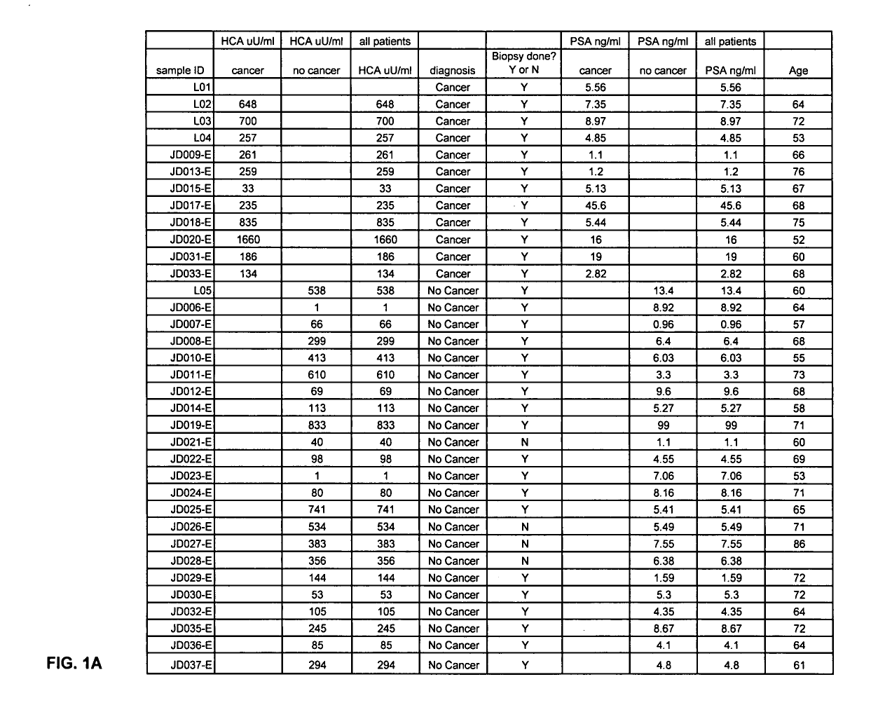

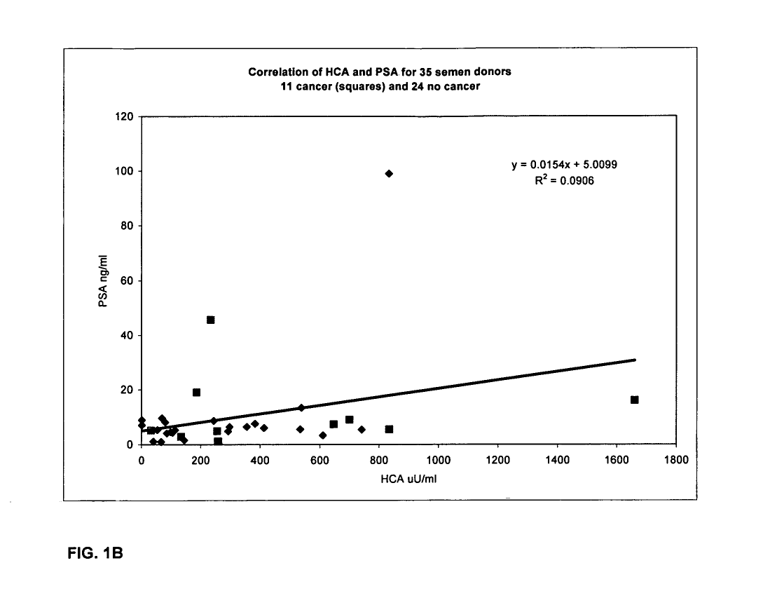

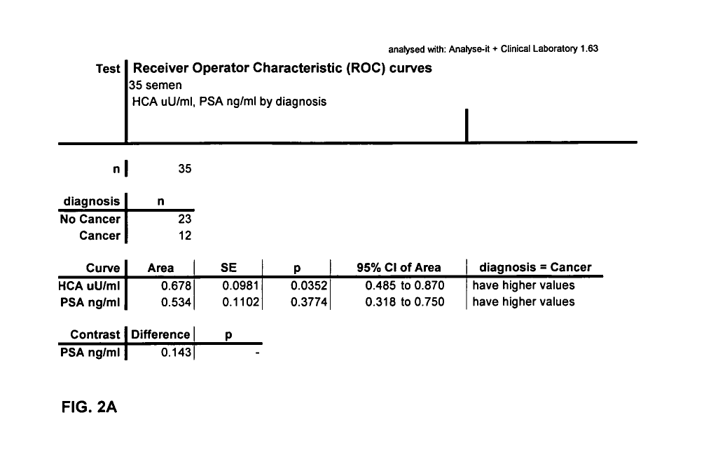

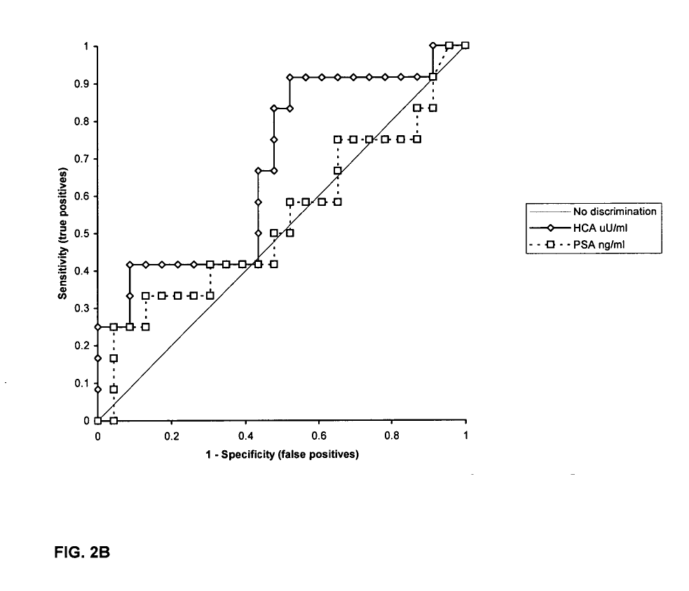

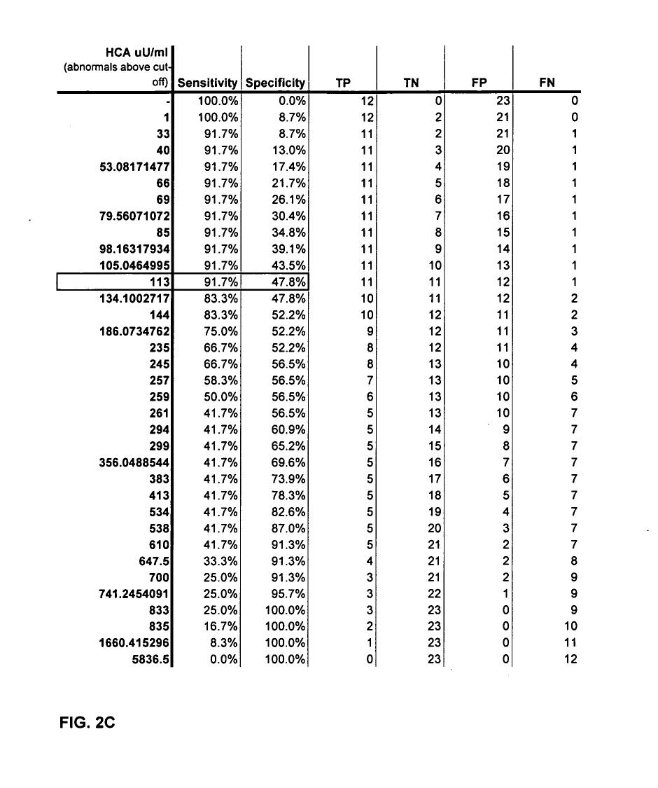

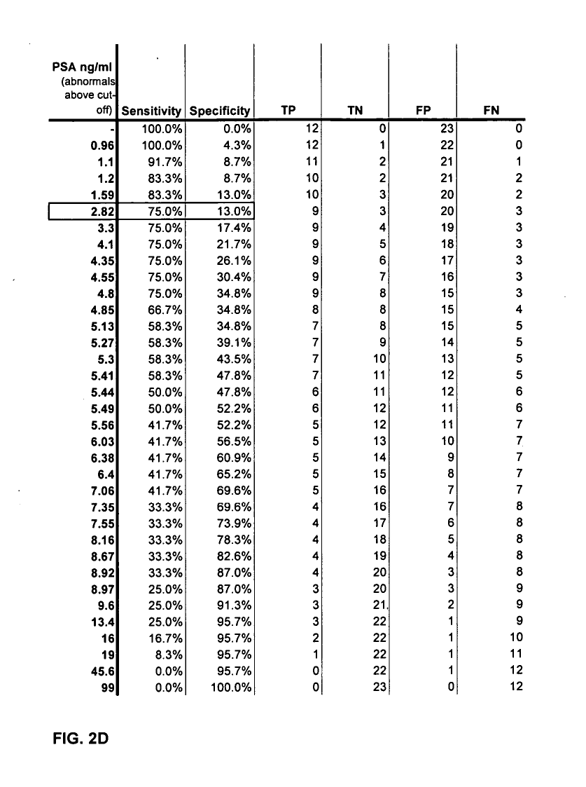

Raw data from the 35 Patient Study are provided in FIG. 1A. The results show that there is no correlation between HCA and PSA values (FIG. 1B). Receiver Operator Characteristic (ROC) curves for HCA and PSA values for the 35 Patient Study are provided in FIG. 2B. The corresponding statistical values for the HCA and PSA determinations are provided in FIG. 2A. The clinical performance (sensitivity, specificity, true positives (TP), true negatives (TN), false positives (FP), false negatives (FN)) of HCA and PSA from this 35 Patient Study are provided in FIGS. 2C and 2D.

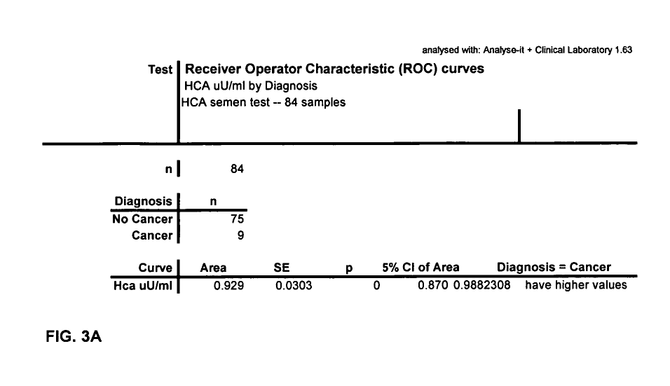

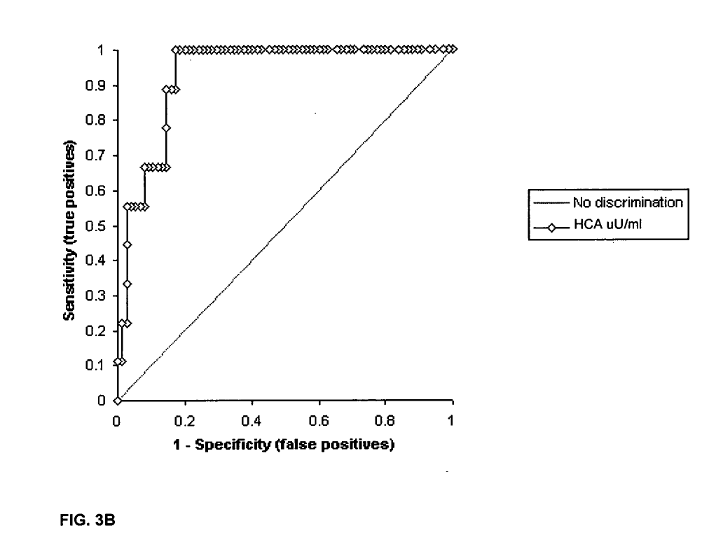

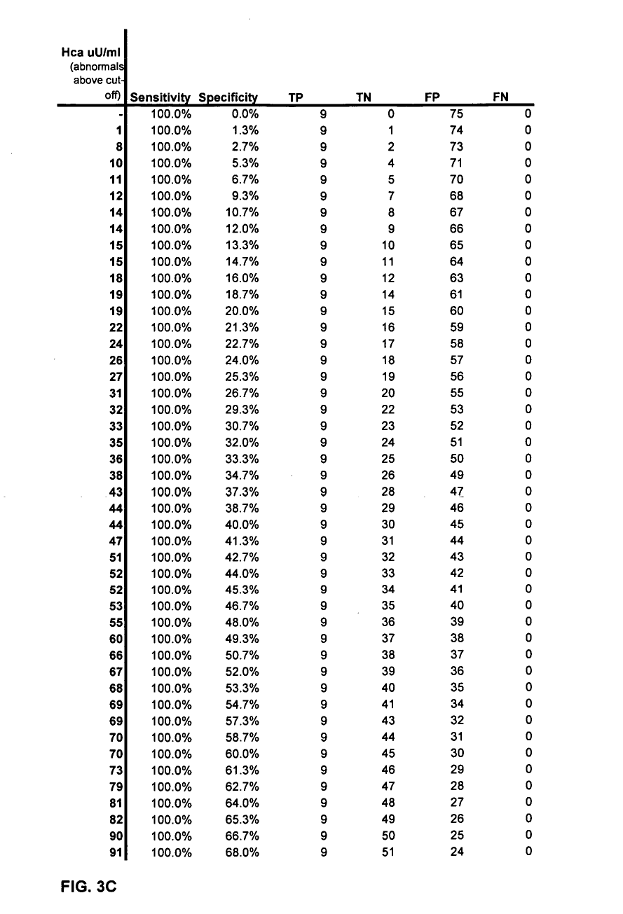

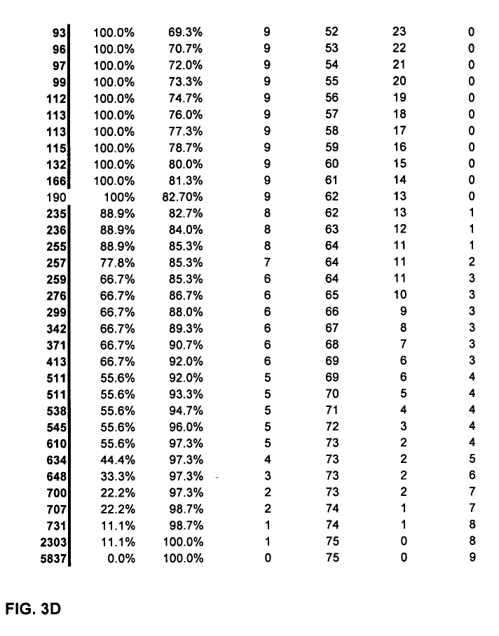

Receiver Operator Characteristic (ROC) curves for HCA and PSA values for the 84 Patient Study are provided in FIG. 3B. The corresponding statistical values for the HCA and PSA determinations are provided in FIG. 3A. The clinical performance (sensitivity, specificity, true positives (TP), true negatives (TN), false positives (FP), false negatives (FN)) of HCA and PSA from this 84 Patient Study are provided in FIGS. 3C and 3D.

### Example 3

### Patient Studies: Analysis of Serum Samples

Two studies on serum samples were conducted. In one study, 433 patient serum samples were analyzed for HCA. In the other study, 358 patient serum samples were analyzed for HCA and PSA.

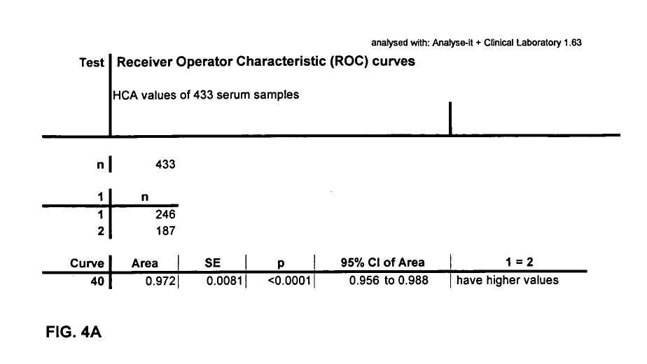

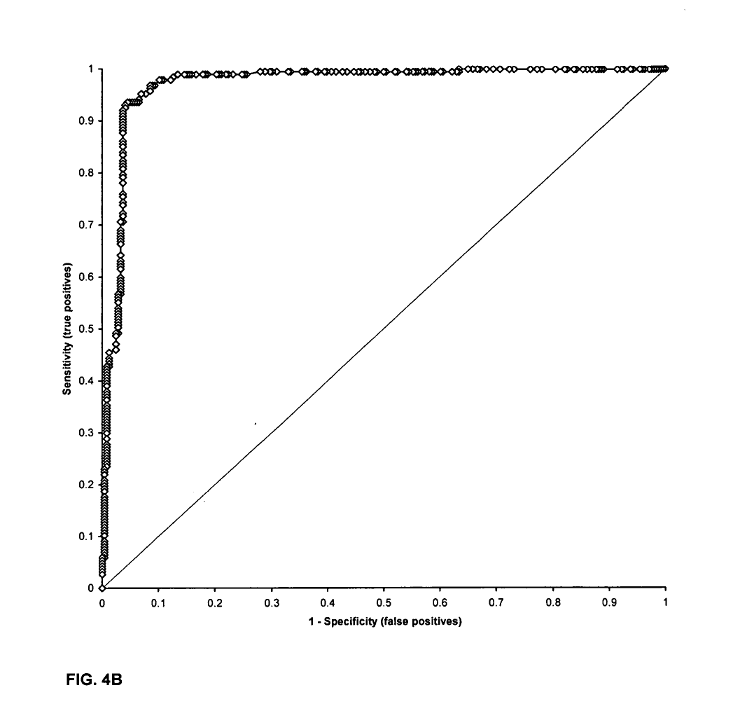

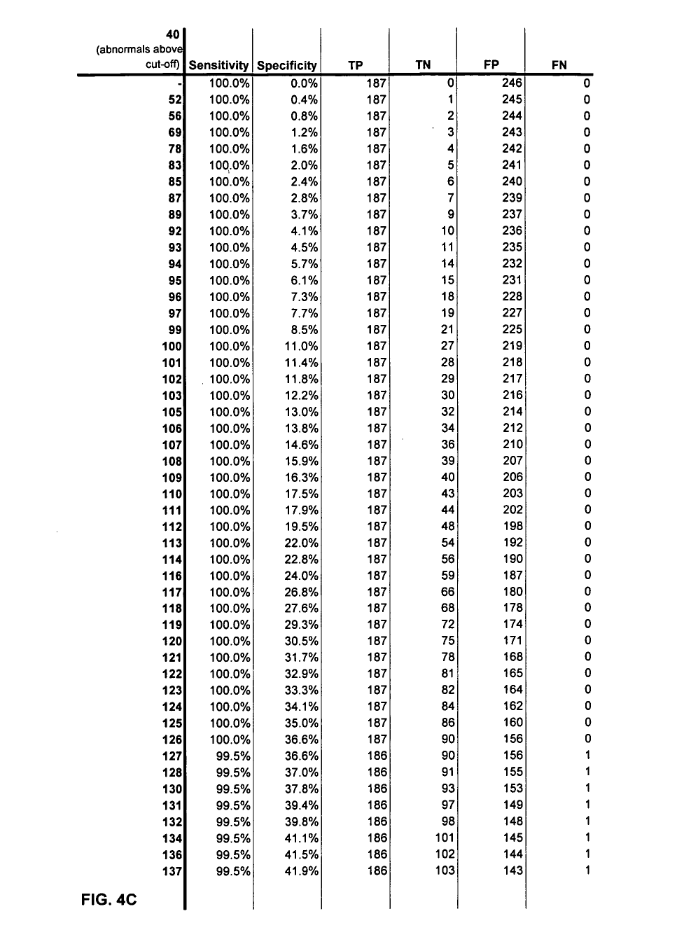

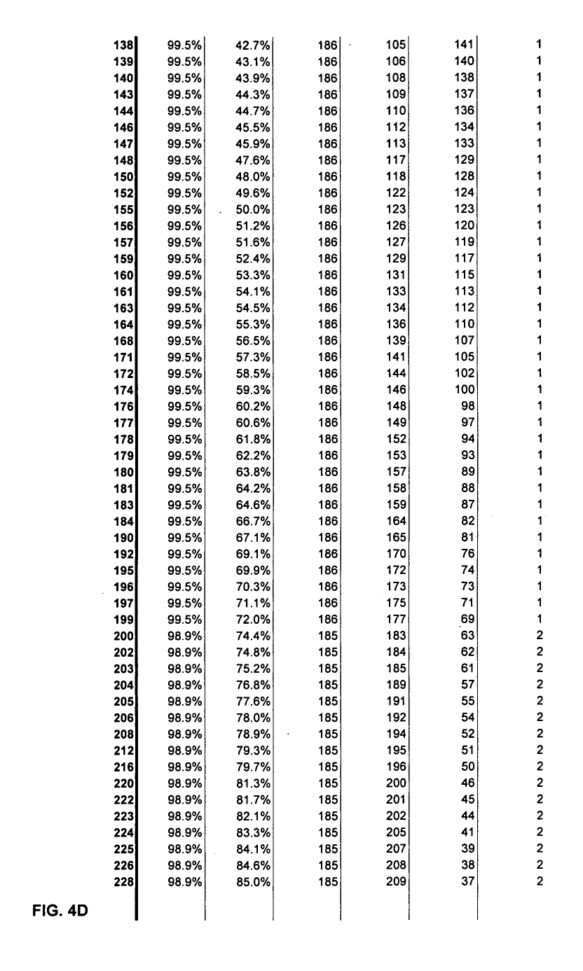

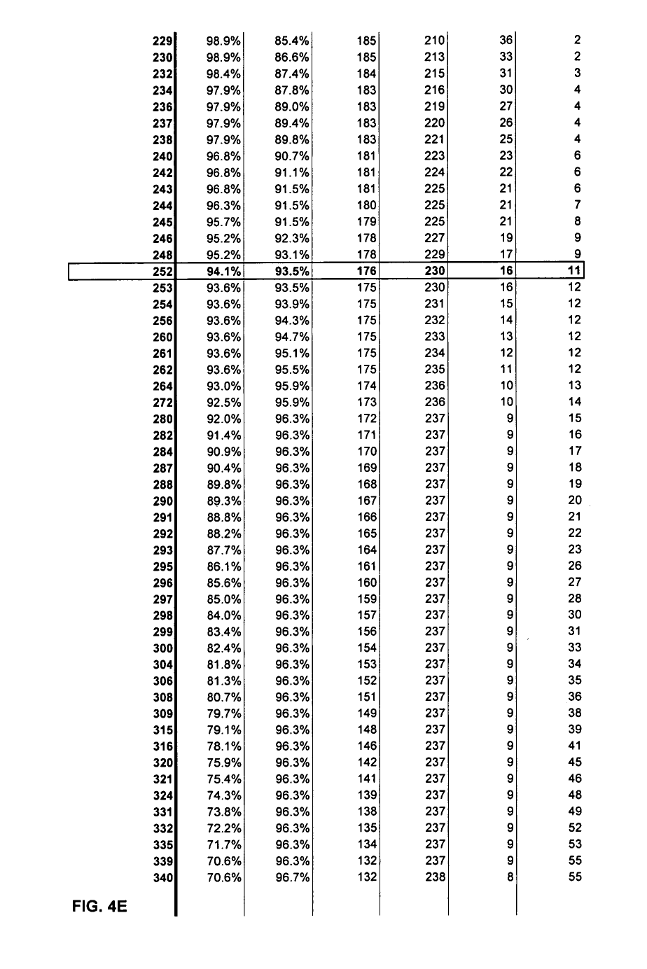

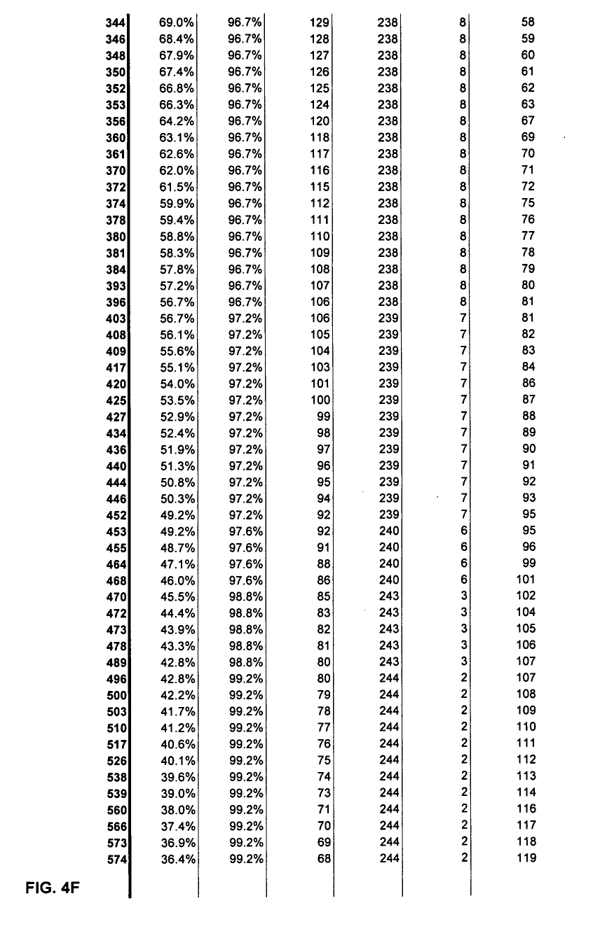

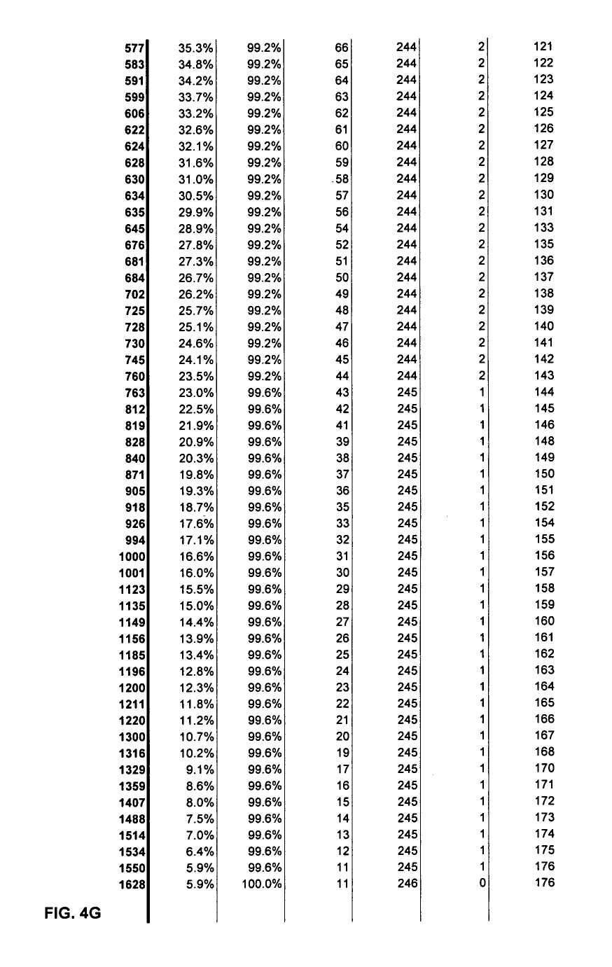

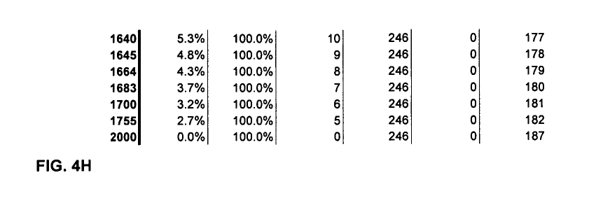

Receiver Operator Characteristic (ROC) curves for HCA values for 433 patient serum samples are provided in FIG. 4B. The corresponding statistical values for the HCA determinations are provided in FIG. 4A. The clinical performance (sensitivity, specificity, true positives (TP), true negatives (TN), false positives (FP), false negatives (FN)) of HCA for the 433 serum samples are provided in FIGS. 4C through 4H.

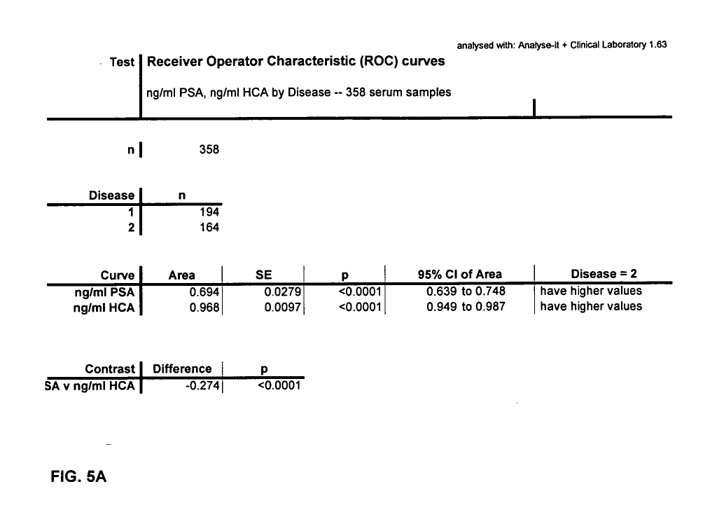

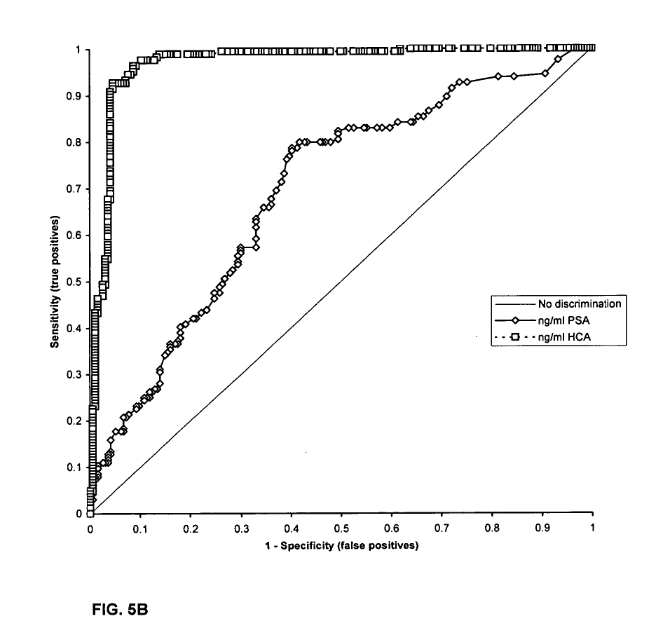

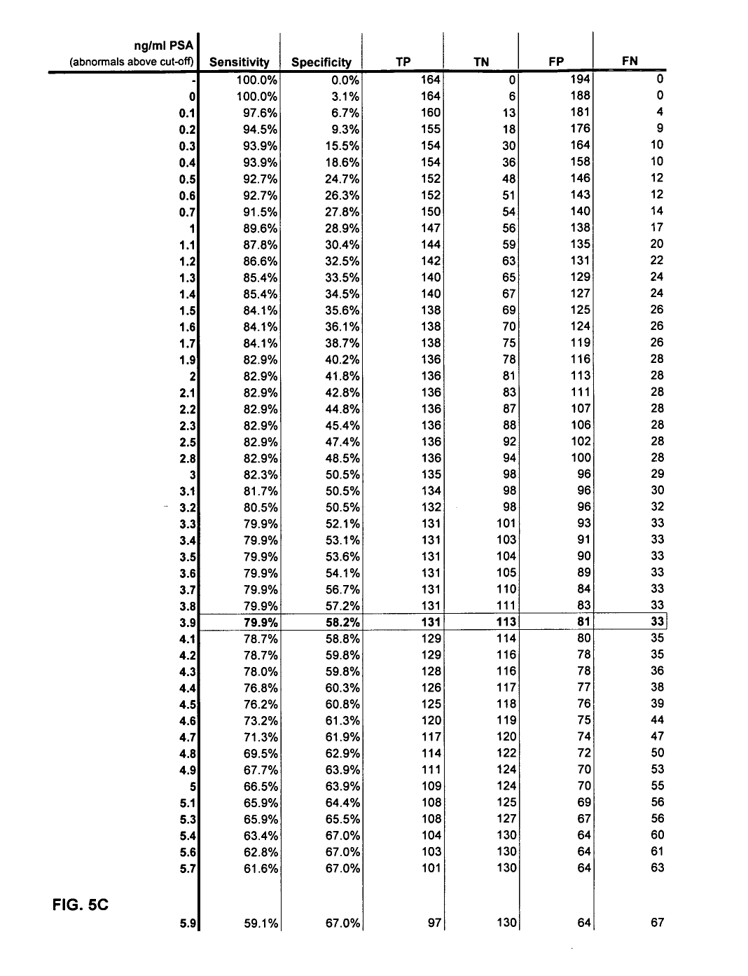

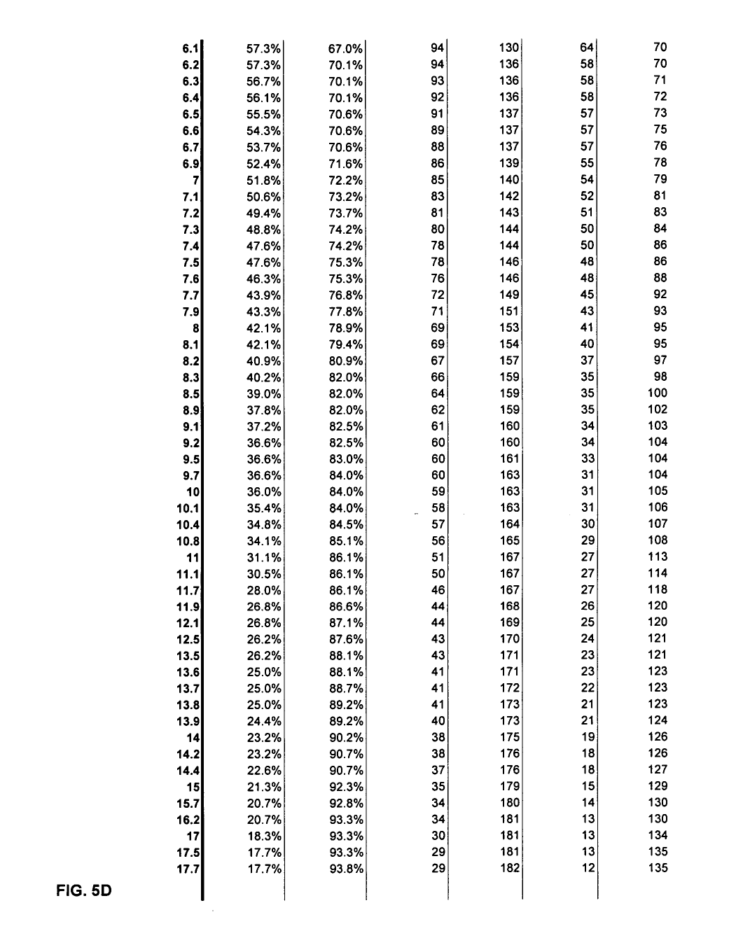

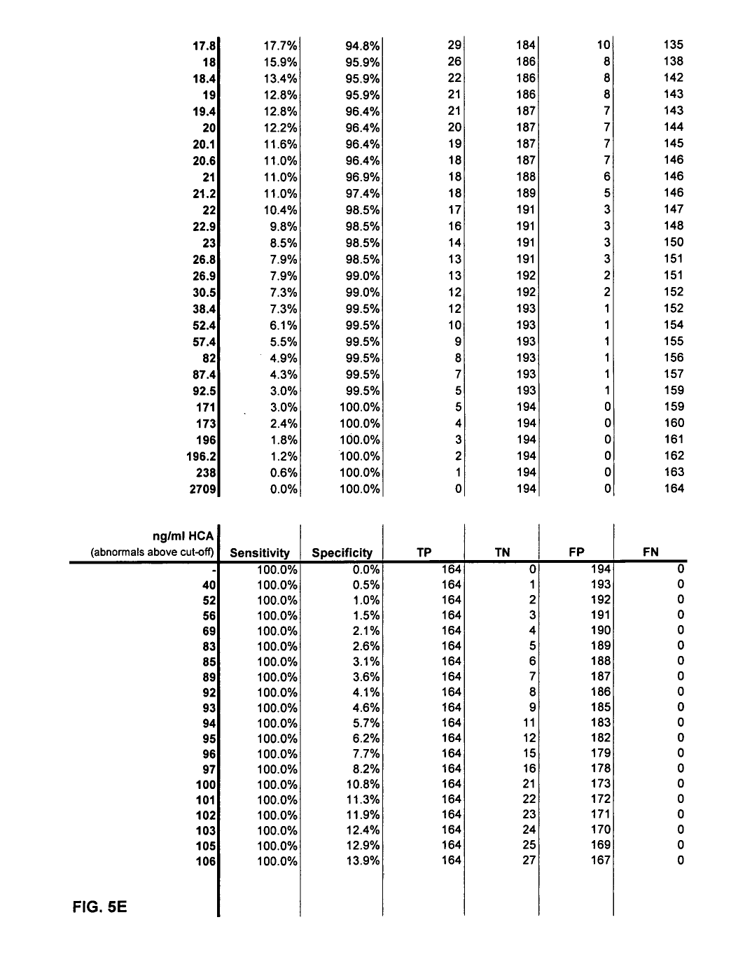

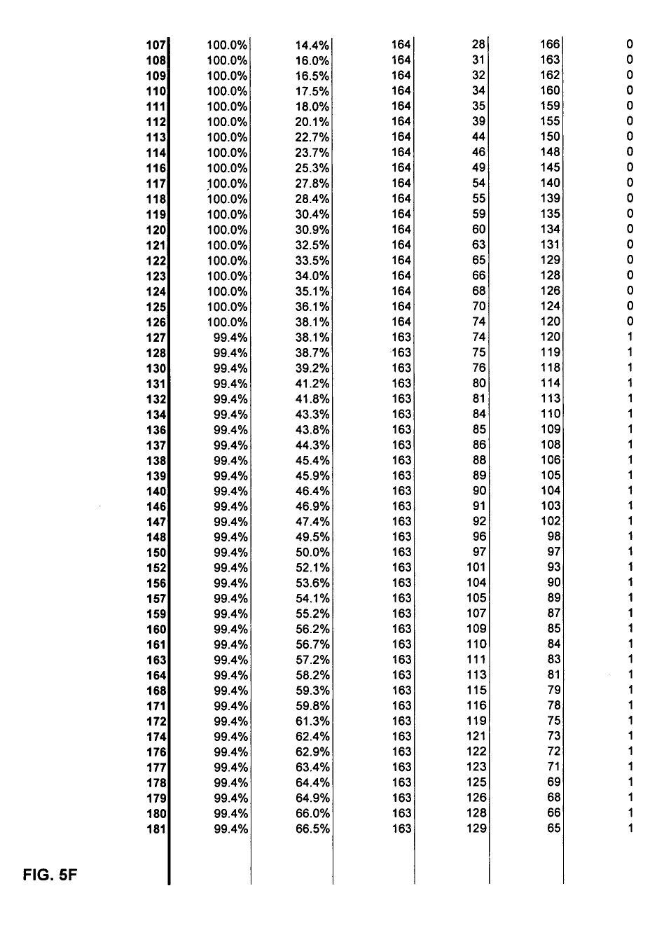

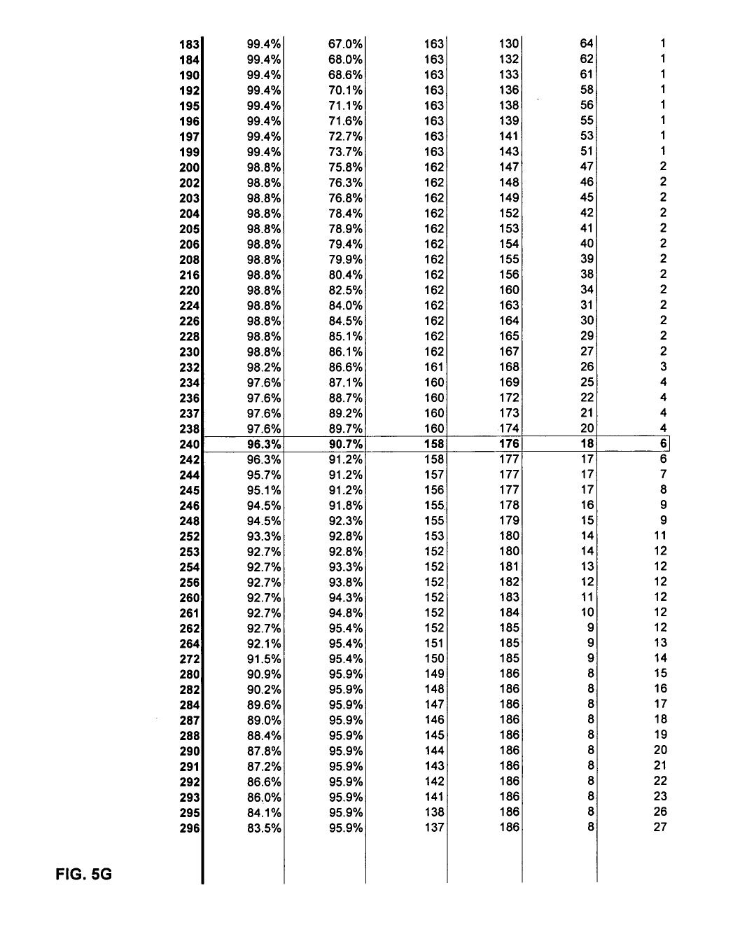

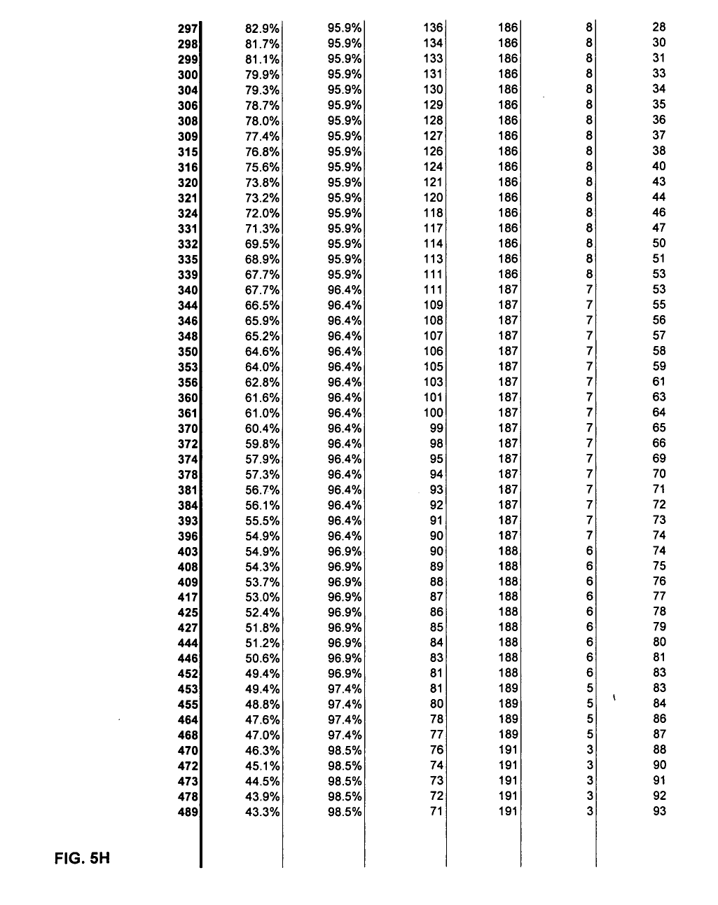

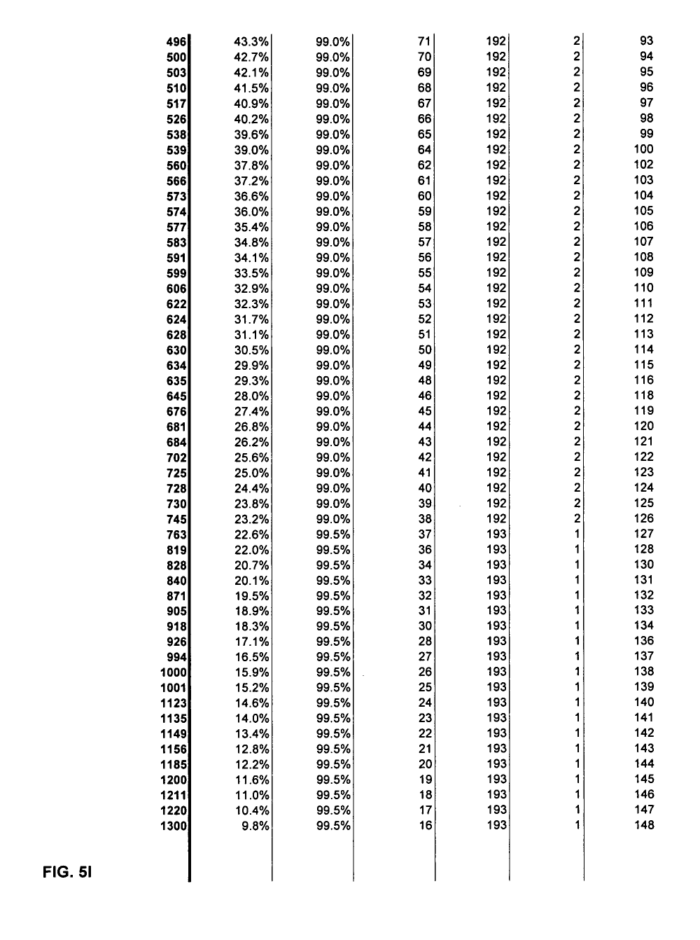

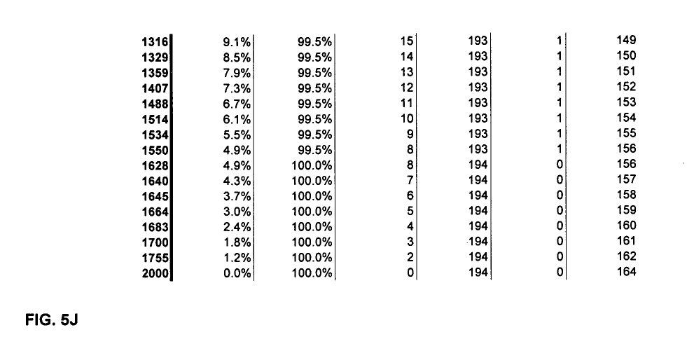

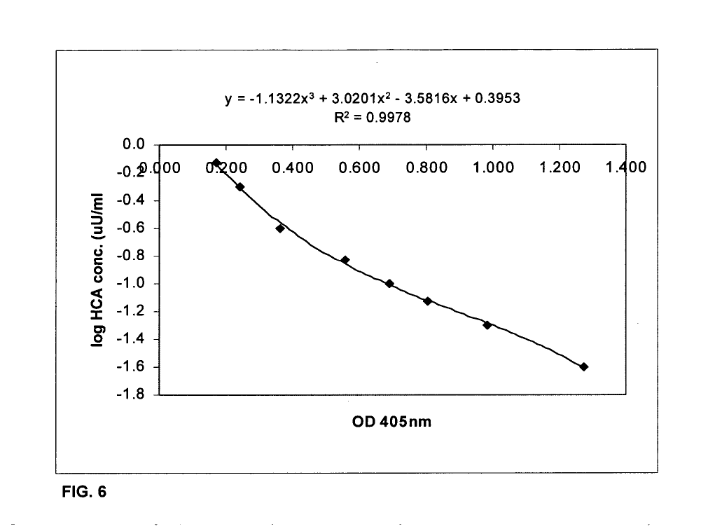

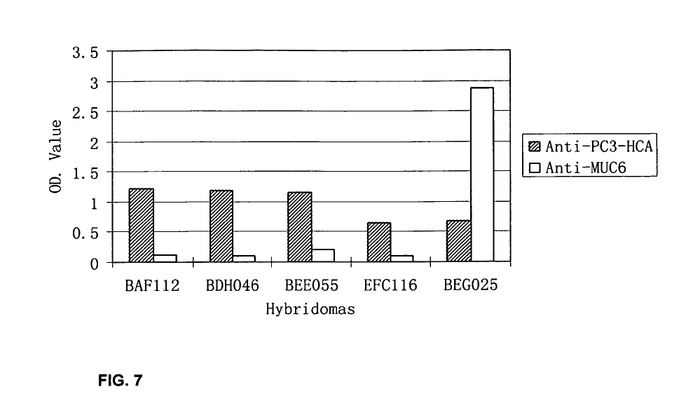

Receiver Operator Characteristic (ROC) curves for PSA and HCA values for 358 serum patient serum samples are provided in FIG. 5B. The corresponding statistical values for the PSA and HCA determinations are provided in FIG. 5A. The clinical performance (sensitivity, specificity, true positives (TP), true negatives (TN), false positives (FP), false negatives (FN)) of HCA for the 358 serum samples are provided in FIGS. 5C through 4J.

### Example 4

### Method for Improvement of HCA/Epiglycanin (EPGN) Purification

New chromatography procedures for high recovery and purity of HCA/EPGN antigen were developed. EPGN from ascetic fluid of TA3 MM1 cell line and the culture supernatant from the A549 cell line were purified using a AE3-HRP coupled affinity column and a MONO Q anion exchange column. More purified material with a higher recovery was achieved. To minimize HCA precipitation and improve HCA recovery, urea and/or CHAPS can be used for elution. The purity and specificity of HCA/EPGN can be improved by anion exchange chromatography followed by affinity chromatography or by affinity chromatography alone compared with the size exclusion chromatography.

### Example 5

### HCA Purification from Prostate Cancer Cell Lines and from Prostate Cancer Tissues

HCA Purification from Prostate Cancer Cell Lines

Several prostate cancer cell lines including PC3 were screened. PC3 prostate cancer cells showed HCA expression. Thus, the PC3 cell line was used for purification of HCA.

HCA was purified from the PC3 cell line using size exclusion chromatography and anion exchange chromatography followed by affinity chromatography with elution by high pH buffer. HCA was concentrated using a lyophilizer. PC3 provides a source of unlimited HCA antigen for assays and for use in the generation of new monoclonal antibodies.

HCA Purification from Prostate Cancer Tissues

HCA purification from prostate tumor tissues was carried out using several preparation protocols, including physical homogenization, chemical treatment (such as with CHAPS detergent and guanidinium chloride), and collagenase treatment.

### Example 6

### Cross-Reactive Antigen MUC6

A cross-reactive species in the lumens of seminal vesicles has been discovered. This species, MUC6, is cross-reactive with the anti-HCA antibody AE3. Results from histochemistry studies show that seminal vesicles from cancer patients were stained by both AE3 and NCL MUC6 antibodies. However, the prostate tissues from the same patients were stained with AE3 antibody and not with the anti-MUC6 antibody. These results demonstrate that the specificity of HCA to the cancerous prostate organ, and that MUC6 arises from the seminal vesicles and is not typically associated with prostate cancer.

To block MUC6 from binding with an anti-HCA antibody in the sample immunoassay, an agonist or antagonist that blocks or interferes with MUC6 binding to anti-HCA antibodies, but does not block or interfere with HCA binding to anti-HCA antibodies, can be added to the sample before or at the same time the assay is performed. In a particular embodiment, MUC6 binding can be blocked by adding an anti-MUC6 antibody to the sample before or at the same time the sample competition assay is performed. An example of an anti-MUC6 antibody is NCL MUC6 from Vector Laboratories, Inc. (Burlingame, Calif.). Other MUC6 antagonists and agonists, including other anti-MUC6 antibodies, are known and described in the art. In another embodiment, anti-HCA antibodies that are not cross-reactive with MUC6 can be used in immunoassays for detecting HCA. Such antibodies can be generated using HCA isolated either from prostate cancer tissues or from prostate tumor cell lines.

### Example 7

### Monoclonal Antibodies With Low Crossreactivity to MUC6

Antibodies were generated by immunizations with HCA purified from the PC3 prostate cancer cell line. Binding of selected monoclonal antibodies with PC3-HCA and MUC6 were determined by ELISA. The testing plates were coated with 2 μU/ml PC3—HCA and 1:100 diluted MUC6, respectively and supernatants of selected hybridomas were tested by standard ELISA protocol. The monoclonal antibody BEG025, which has strong crossreactivity with MUC6, was used as a positive control.

All publications, patent and patent applications mentioned in this specification are incorporated herein by reference to the same extent as if each individual publication, patent or patent application was specifically and individually incorporated by reference.

While this invention has been particularly shown and described with references to preferred embodiments thereof, it will be understood by those skilled in the art that various changes in form and details may be made therein without departing from the scope of the invention encompassed by the appended claims.

1. A method for diagnosis of prostate cancer in a human subject comprising:
a) determining the level of HCA in a semen sample from said subject; and
b) comparing the level so determined to the level of HCA in a control semen sample,
wherein the presence of an elevated level of HCA in the semen sample from said subject relative to the control is indicative of prostate cancer in said subject.

2. The method of claim 1, wherein the level of HCA is determined by performing an immunoassay.

3. The method of claim 2, wherein the immunoassay is selected from the group consisting of: a competitive immunoassay and a sandwich immunoassay.

4. A method for diagnosis of prostate cancer in a human subject comprising:
a) contacting a semen sample from said subject with an antibody which binds to HCA or an antigen binding fragment thereof;
b) assaying for binding of HCA to said antibody or antigen-binding fragment;
c) determining the level of HCA bound to said antibody or antigen-binding fragment; and
d) comparing the level of bound HCA determined in step c) to the level of HCA bound to said antibody in a control semen sample,
wherein the presence of an elevated level of bound HCA determined in step c) relative to the control is indicative of prostate cancer in said subject.

5. The method of claim 4, wherein binding of HCA to said antibody or antigen-binding fragment is assayed by performing an immunoassay.

6. The method of claim 5, wherein the immunoassay is selected from the group consisting of: a competitive immunoassay and a sandwich immunoassay.

7. The method of claim 6, wherein binding of HCA to said antibody or antigen-binding fragment is assayed by a competitive immunoassay comprising contacting said antibody or antigen binding fragment with a competing antigen that is immunologically cross-reactive with HCA.

8. The method of claim 6, wherein binding of HCA to said antibody or antigen-binding fragment is assayed by a sandwich immunoassay in which said antibody or antigen-binding fragment is a first antibody or antigen-binding fragment and binding of HCA to said first antibody or antigen-binding fragment is determined by contacting a second antibody or antigen-binding fragment thereof that binds to HCA.
